(19) United States
(12) Patent Application Publication
(10) Pub. No.: US 2019/0311802 A1
(43) Pub. Date: Oct. 10, 2019

(54) SURGICAL INSTRUMENT MANAGEMENT SYSTEM

(71) Applicant: DGSHAPE Corporation, Hamamatsu-shi (JP)

(72) Inventors: Takaaki KOKUBO, Hamamatsu-shi (JP); Masaki HANAJIMA, Hamamatsu-shi (JP)

(21) Appl. No.: 16/378,623

(22) Filed: Apr. 9, 2019

(30) Foreign Application Priority Data
Apr. 10, 2018 (JP) 2018-075207

(51) Int. Cl. G16H 40/63; A61B 90/90; A61B 90/00

(52) U.S. Cl. CPC G16H 40/63; A61B 90/90; A61B 2090/081; A61B 2090/0803; A61B 90/08

(57) ABSTRACT

A surgical instrument management system includes a reader that reads an identification mark, and a management terminal that manages predetermined information associated with the identification mark, the predetermined information including a state of use of a surgical instrument. The management terminal includes a storage that stores the predetermined information and a predetermined discard condition under which the surgical instrument is to be discarded, an updater that updates the state of use of the surgical instrument, a first determiner that determines whether or not the state of use of the surgical instrument meets the discard condition, and a first notifier that, in the case where the first determiner determines that the state of use of the surgical instrument meets the discard condition, notifies that the surgical instrument needs to be discarded.

### CROSS REFERENCE TO RELATED APPLICATION

This application claims the benefit of priority to Japanese Patent Application No. 2018-075207 filed on Apr. 10, 2018. The entire contents of this application are hereby incorporated herein by reference.

### BACKGROUND OF THE INVENTION

### 1. Field of the Invention

The present invention relates to a surgical instrument management system.

### 2. Description of the Related Art

Conventionally, a plurality of surgical instruments including forceps and the like are used to perform a surgical operation in a hospital. Such surgical instruments include a surgical instrument for so-called “single-time use”, which is permitted to be used only once, and a surgical instrument permitted to be used a predetermined number of times by being, for example, disinfected or sterilized. An expiration time for use is set for some of the surgical instruments. As can be seen, the surgical instruments are restricted regarding the use thereof in some way or another.

Usually, surgical instruments are used repeatedly in a circulation cycle described below. The circulation cycle includes a surgical operation step, a collection step, a cleaning step, an assembly step, a sterilization step, and a storage step. In the surgical operation step, a surgical operation is performed by use of the surgical instruments. In the collection step, the surgical instruments used for the surgical operation are collected. In the cleaning step, the collected surgical instruments are cleaned. In the assembly step, the cleaned surgical instruments are assembled into a surgical instrument set. In the sterilization step, the assembled surgical instrument set is sterilized. In the storage step, the sterilized surgical instrument set is stored.

As described above, the surgical instruments are used repeatedly in the circulation cycle, and need to be managed properly in each of a plurality of hospitals.

There are a huge number of types of surgical instruments, and the total number of the surgical instruments is enormous. Especially, a hospital of a larger scale tends to possess a larger number of types of, and a larger total number of, surgical instruments. It is not easy to manage such a large number of surgical instruments properly. In the case where the surgical instruments are not properly managed, it is possible that, for example, a surgical instrument that has already been used a number of times equal to the limit of the number of times of use, is used for a surgical operation. It is also possible that, for example, a surgical instrument that has not been used a number of times equal to the limit of the number of times of use is inadvertently discarded.

### SUMMARY OF THE INVENTION

Preferred embodiments of the present invention provide surgical instrument management systems that manage surgical instruments properly.

A surgical instrument management system according to a preferred embodiment of the present invention manages a state of use of a surgical instrument. The surgical instrument is usable repeatedly in a predetermined circulation cycle including performing a surgical operation by use of the surgical instrument, collecting the surgical instrument used for the surgical operation, cleaning the collected surgical instrument, assembling a surgical instrument set by use of the surgical instrument, sterilizing the assembled surgical instrument set, and storing the sterilized surgical instrument set. The surgical instrument is provided with an identification mark. The surgical instrument management system includes a reader that reads the identification mark, and a management terminal that manages predetermined information associated with the identification mark, the predetermined information including the state of use of the surgical instrument. The management terminal includes a storage that stores the predetermined information and a predetermined discard condition under which the surgical instrument is to be discarded, an updater that updates the state of use of the surgical instrument, a first determiner that determines whether or not the state of use of the surgical instrument meets the discard condition, and a first notifier that, in the case where the first determiner determines that the state of use of the surgical instrument meets the discard condition, notifies that the surgical instrument needs to be discarded.

With a surgical instrument management system according to a preferred embodiment of the present invention, the updater of the management terminal updates the state of use of the surgical instrument provided with the identification mark. The first determiner of the management terminal determines whether or not the state of use of the surgical instrument meets the discard condition under which the surgical instrument is to be discarded. In the case where the first determiner determines that the state of use of the surgical instrument meets the discard condition, the first notifier of the management terminal notifies that the surgical instrument needs to be discarded. In this manner, the state of use of the surgical instrument is updated at a predetermined timing by the management terminal. In the case where it is determined that the surgical instrument needs to be discarded based on the state of use thereof, it is notified that the surgical instrument needs to be discarded. Therefore, the operator needs to discard only the notified surgical instrument so as to prevent the operator forgetting discarding the surgical instrument to be discarded or discarding the surgical instrument that is still usable. The so-called “single-time use” surgical instrument is opened immediately before the surgical operation and is not provided with any identification mark. Therefore, when, for example, a certain surgical instrument is found not to be provided with any identification mark when the reader attempts to read the identification mark in any of the steps of the circulation cycle (excluding the surgical operation step), the operator may recognize that this surgical instrument is for “single-time use” and thus discard this surgical instrument.

Preferred embodiments of the present invention provide surgical instrument management systems that manage surgical instruments properly.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic view showing a surgical instrument management system according to a preferred embodiment of the present invention.

FIG. 2 illustrates a circulation cycle in which a surgical instrument management system according to a preferred embodiment of the present invention is to be used.

FIG. 3 is a block diagram showing a structure of a management terminal according to a preferred embodiment of the present invention.

FIG. 4 shows a state of a display device of a management terminal, where a surgical instrument is displayed.

FIG. 5 shows a state of a display device of a management terminal, where a warning regarding a limit of a number of times of use is displayed.

FIG. 6 shows a state of a display device of a management terminal, where it is indicated that a displayed surgical instrument is prohibited from being used.

FIG. 7 shows a state of a display device of a management terminal, where it is indicated that a displayed surgical instrument needs to be discarded.

FIG. 8 shows a state of a display device of a management terminal, where another surgical instrument is displayed.

FIG. 9 shows a state of a display device of a management terminal, where a warning regarding an inspection is displayed.

FIG. 10 shows a state of a display device of a management terminal, where it is indicated that a displayed surgical instrument is prohibited from being used.

FIG. 11 shows a state of a display device of a management terminal, where it is indicated that a displayed surgical instrument needs to be inspected.

FIG. 12 shows a state of a display device of a management terminal, where still another surgical instrument is displayed.

FIG. 13 shows a state of a display device of a management terminal, where it is proposed to change a displayed surgical instrument.

FIG. 14 shows a state of a display device of a management terminal, where a list of surgical instruments is displayed.

FIG. 15 shows a state of a display device of a management terminal, where a post-change surgical instrument is displayed.

### DETAILED DESCRIPTION OF THE PREFERRED EMBODIMENTS

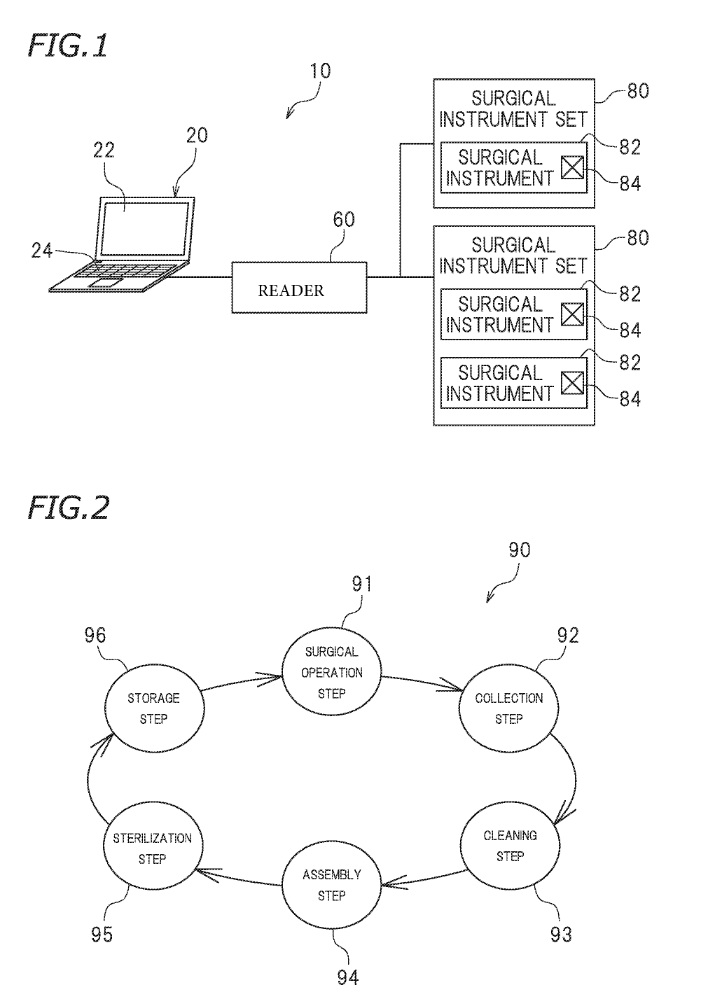

Hereinafter, preferred embodiments of surgical instrument management systems according to the present invention will be described with reference to the drawings. A surgical instrument management system 10 manages a state of use (e.g., number of times of use) of a surgical instrument 82. As shown in FIG. 1, the surgical instrument management system 10 includes a management terminal 20, a reader 60 and a plurality of surgical instruments 82. The management terminal 20 and the reader 60 are communicably connected with each other in a wired manner. The management terminal 20 and the reader 60 may be communicably connected with each other wirelessly. The management terminal 20 manages predetermined information that is associated with an identification mark 84 read by the reader 60 and that includes a state of use of the surgical instrument 82 corresponding to the identification mark 84. The management terminal 20 in this preferred embodiment is a notebook personal computer. Alternatively, the management terminal 20 may be a tablet terminal, a smartphone, a desktop personal computer or the like.

Examples of the surgical instrument 82 include forceps, surgical scissors, scalpels (e.g., laser scalpels and electric scalpels), ultrasonic cutters, scalpel holders, cannulae, tweezers, retractors, scales, probes, elevators, raspatories, siphons, rib spreaders, rib contractors, needle holders, injectors, metal bowls, kidney basins, cups, pins, mirrors, files, mouth gags, clamps, handpieces, elevators (elepatriums), chisels, osteotrites, raspatories, mirrors, suture needles, punches (stanze), water receiving basins, needles, (tongue) pressors, bougies, vent pipes, bone impactors, luer rongeurs, radio pliers, hammers, goniometers, fraises, droppers, metal swabs, enemators, syringes, endoscopes, and the like. The above lists merely include examples of the surgical instrument 82, and the surgical instrument 82 is not limited to any of the above examples. The surgical instruments 82 include an instrument usable repeatedly a predetermined number of times or within a predetermined time period in a predetermined circulation cycle 90 described below, and an instrument usable only once in a surgical operation step 91 described below (hereinafter, such a surgical instrument will be referred to as a “single-time use instrument”).

As shown in FIG. 2, the circulation cycle 90 includes the surgical operation step 91, a collection step 92, a cleaning step 93, an assembly step 94, a sterilization step 95, and a storage step 96. In the circulation cycle 90, the surgical instrument 82 circulates in the order of the surgical operation step 91, the collection step 92, the cleaning step 93, the assembly step 94, the sterilization step 95, the storage step 96, and again, the surgical operation step 91. In the circulation cycle 90, a surgical instrument set 80 including one or a plurality of the surgical instruments 82 circulates. The specifics of the surgical instrument set 80 are different in accordance with the contents of the surgical operation or the doctor using the surgical instrument set 80, and there are a variety of surgical instrument sets 80.

In the surgical operation step 91, a surgical operation is performed on a patient by the use of the surgical instruments 82 in the stored surgical instrument set 80. In the collection step 92, the surgical instruments 82 used for the surgical operation are collected. Usually in the collection step 92, the surgical instruments 82 are collected separately, not in the form of the surgical instrument set 80. In the cleaning step 93, the collected surgical instruments 82 are cleaned. In the assembly step 94, the cleaned surgical instruments 82 are assembled into the surgical instrument set 80. More specifically, some of the surgical instruments 82 are cleaned in a state of being divided into individual parts in the cleaning step 93, and these individual parts are assembled into the respective surgical instruments 82. Also in the assembly step 94, the surgical instruments 82 cleaned separately in the cleaning step 93 are returned so as to be positionally arranged properly in the predetermined surgical instrument set 80. In the sterilization step 95, the surgical instruments 82 in the above-described assembled state and/or in the above-described positionally arranged state are sterilized. Methods for sterilization include, for example, gas sterilization with ethylene oxide gas, plasma sterilization, thermal sterilization by use of an autoclave, and the like. In the storage step 96, the sterilized surgical instrument set 80 is stored in a predetermined storage space. In this specification, the term “assemble” is intended to encompass both of a case where the parts of a surgical instrument are assembled into one surgical instrument, and a case where the plurality of surgical instruments are located properly in the surgical instrument set as described above. The expression “assembled surgical instrument” is intended to encompass both of a surgical instrument in a form of an assembly including the above-described parts, and a surgical instrument located properly in the surgical instrument set as described above.

On each of the surgical instruments 82, the identification mark 84 is provided. The identification mark 84 may be, for example, a two-dimensional code, a code of a GS1 coding system (e.g., numerical figure of 26 digits), an IC tag, or the like. The identification mark 84 is associated with predetermined information on the surgical instrument 82, including the state of use. The predetermined information may be, for example, the name of the surgical instrument 82, a limit of the number of times of use of the surgical instrument 82, a number of times the surgical instrument 82 has already been used (hereinafter, may be referred to as the “number of times of use of the surgical instrument 82” or the “number of times of use”), a limit of the number of times of sterilization of the surgical instrument 82, a number of times the surgical instrument 82 has already been sterilized (hereinafter, may be referred to as the “number of times of sterilization of the surgical instrument 82” or the “number of times of sterilization”), a usable time period of the surgical instrument 82, an elapsed time period after the start of use of the surgical instrument 82 (hereinafter, may be referred to as the “elapsed time period of the surgical instrument 82” or the “elapsed time period”), a name of the surgical instrument set 80 including the surgical instrument 82, a site at which the surgical instrument 82 is stored, an ID of the surgical instrument 82, a date/time when the surgical instrument 82 was used, a name of the operator who used the surgical instrument 82, or the like. The number of times of use of the surgical instrument 82, the number of times of sterilization of the surgical instrument 82, and the elapsed time period after the start of use of the surgical instrument 82 are included in the state of use of the surgical instrument 82. The “limit of the number of times of use of the surgical instrument 82” is the number of times the surgical instrument 82 is permitted to be used in the surgical operation step 91. The “limit of the number of times of sterilization of the surgical instrument 82” is the number of times the surgical instrument 82 (surgical instrument set 80) is permitted to be sterilized in the sterilization step 95. In this preferred embodiment, the “start of use of the surgical instrument 82” refers to the time point at which the surgical instrument 82 is registered in the management terminal 20. Alternatively, the “start of use of the surgical instrument 82” may refer to the time point at which the assembly of the surgical instrument set 80 is finished in the assembly step 94. The “usable time period of the surgical instrument 82” is the time period in which the surgical instrument 82 is usable after the start of use thereof. The limit of the number of times of use of the surgical instrument 82, the limit of the number of times of sterilization of the surgical instrument 82, and the usable time period of the surgical instrument 82 vary in accordance with the type of the surgical instrument 82. There is a surgical instrument 82 for which the limit of the number of times of use, the limit of the number of times of sterilization, and the usable time period are all set. There is also a surgical instrument 82 for which either one of the limit of the number of times of use, the limit of the number of times of sterilization, and the usable time period is set. The single-time use instrument is usually used for the first time in the surgical operation step 91, and therefore, has no identification mark 84 provided thereon. For this reason, when, for example, the reader 60 attempts to read the identification mark 84 in the assembly step 94, the surgical instrument 82 with no identification mark 84 is recognized as a surgical instrument to be discarded as a single-time use instrument. In this specification, the expression “expiration time for use” used regarding the surgical instrument 82 refers to the time point until which the surgical instrument 82 is permitted to be used. An expression that the expiration time for use of one surgical instrument 82 is closer than the expiration time for use of another surgical instrument 82 indicates that the elapsed time period of the one surgical instrument 82 is longer than the elapsed time period of the another surgical instrument 82.

The reader 60 reads the identification mark 84 provided on the surgical instrument 82. The reader 60 transmits information on the identification mark 84 to the management terminal 20. The reader 60 may be any device capable of reading the identification mark 84 with no specific limitation. In the case where, for example, the identification mark 84 is a two-dimensional code, the reader 60 may be a two-dimensional code reader capable of reading a two-dimensional code. In the case where the identification mark 84 is an IC tag, the reader 60 may be an IC tag reader. The act of the reader 60 reading the identification mark 84 is performed in, for example, at least one step in the circulation cycle 90 described above (e.g., in the assembly step 94).

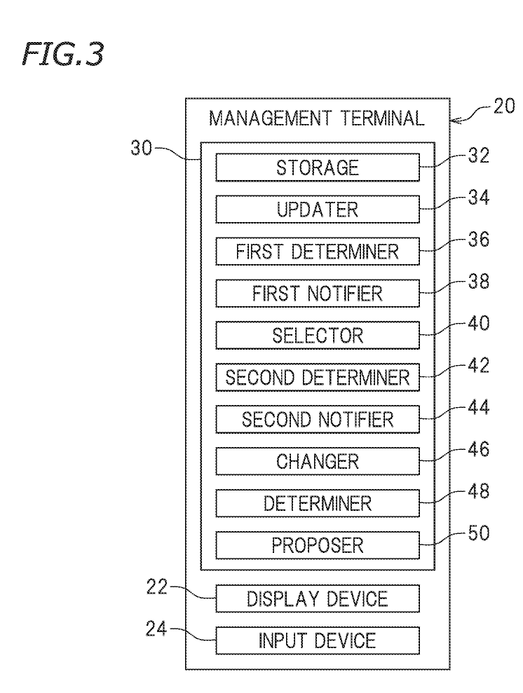

Now, a detailed structure of the management terminal 20 will be described. The management terminal 20 includes a built-in computer. As shown in FIG. 3, the management terminal 20 includes a controller 30 executing a predetermined process in accordance with a program, a display device 22 including a display or the like, and an input device 24 including a keyboard, a touch panel, a mouse or the like. The controller 30 is configured or programmed to include a storage 32, an updater 34, a first determiner 36, a first notifier 38, a selector 40, a second determiner 42, a second notifier 44, a changer 46, a determiner 48, and a proposer 50. These elements may be provided in the form of software. Namely, these elements may be realized by the controller 30 (computer) as a result of a computer program being read into the controller 30. The computer program may be provided as being stored on a computer-readable storage medium such as a CD, a DVD or the like. The computer program may be downloadable via the Internet. Alternatively, the above-described elements may be realized by a processor and/or a circuit included in the controller 30. Specific control or the like executed by each of the above-described elements will be described below.

The storage 32 stores the above-described predetermined information including the state of use of each of the plurality of surgical instruments 82 managed by the management terminal 20. The storage 32 stores a predetermined discard condition under which each of the surgical instruments 82 is to be discarded. The discard condition includes, for example, a first condition that the number of times of use of the surgical instrument 82 reached the limit of the number of times of use, a second condition that the number of times of sterilization of the surgical instrument 82 reached the limit of the number of times of sterilization, and a third condition that the elapsed time period after the start of use of the surgical instrument 82 reached the usable time period. The storage 32 also stores a predetermined inspection condition under which each of the surgical instruments 82 is to be inspected. The inspection condition includes, for example, a fourth condition that the number of times of use of the surgical instrument 82 reached a predetermined number of times smaller than the limit of the number of times of use (e.g., half of the limit of the number of times of use); a fifth condition that the number of times of sterilization of the surgical instrument 82 reached a predetermined number of times smaller than the limit of the number of times of sterilization (e.g., half of the limit of the number of times of sterilization), and a sixth condition that the elapsed time period of the surgical instrument 82 reached a predetermined time period shorter than the usable time period (e.g., half of the usable time period).

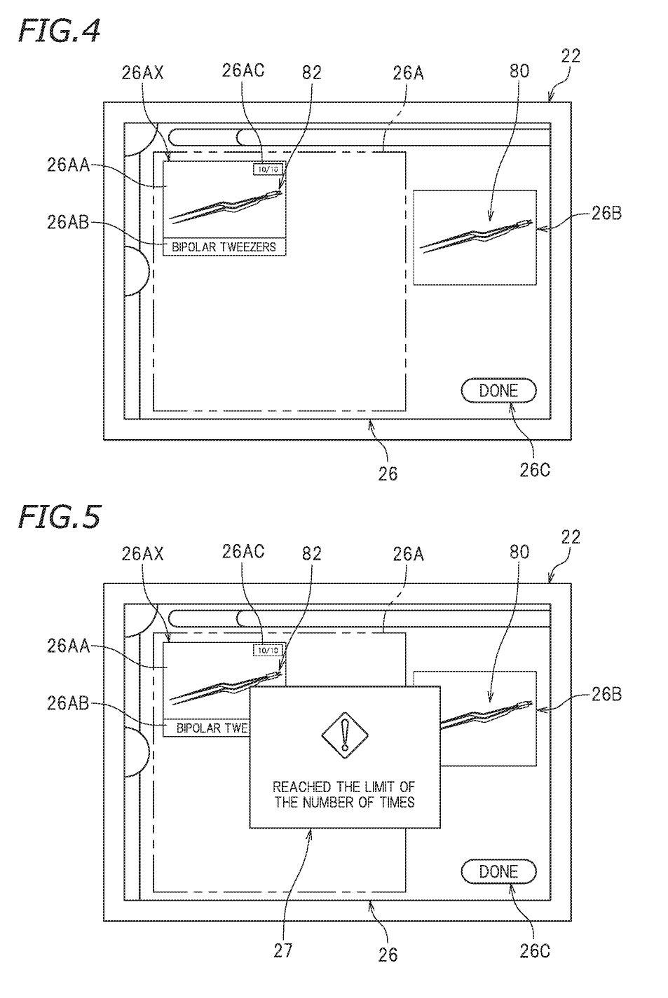

FIG. 4 shows an example of basic operation screen 26 displayed on the display device 22 when the identification mark 84 of a surgical instrument 82 is read by the reader 60. A predetermined application is executed, so that the basic operation screen 26 is displayed on the display device 22. The basic operation screen 26 includes a first region 26A displaying contents of the surgical instrument 82 associated with the identification mark 84 read by the reader 60, a second region 26B displaying contents of the surgical instrument set 80, and a button 26C that is allowed to be pressed when the assembly of the surgical instrument set 80 is finished and indicates, when being pressed, that the assembly of the surgical instrument set 80 is finished. The first region 26A includes a display field 26AX displaying information on each of the surgical instruments 82. The display field 26AX includes a first area 26AA displaying a photograph or an illustration of the surgical instrument 82, a second area 26AB displaying the name of the surgical instrument 82, and a third area 26AC displaying the number of times of use and the limit of the number of times of use of the surgical instrument 82. The number of times of use of the surgical instrument 82 is displayed in a left portion of the third area 26AC. The limit of the number of times of use of the surgical instrument 82 is displayed in a right portion of the third area 26AC. In the case where the surgical instrument 82 is of a certain type, the third area 26AC may display the number of times of sterilization and the limit of the number of times of sterilization, or the elapsed time period and the usable time period. When the identification mark 84 of the surgical instrument 82 is read by the reader 60, the contents of the surgical instrument 82 associated with the identification mark 84 are displayed in the first region 26A. In the second region 26B, a photograph or an illustration of the surgical instrument set 80 is displayed.

The updater 34 updates the predetermined information on each of the surgical instruments 82 stored on the storage 32. The updater 34 updates the state of use of the surgical instrument 82 stored on the storage 32. The updater 34 updates the state of use of the surgical instrument 82 when the identification mark 84 of the surgical instrument 82 is read by the reader 60 in the assembly step 94. For example, when the identification mark 84 of the surgical instrument 82 is read by the reader 60 in the assembly step 94, the updater 34 updates the number of times of use of the surgical instrument 82. Namely, the updater 34 adds “1” to the number of times of use. When the reader 60 reads the identification mark 84 for the first time (i.e., when the surgical instrument 82 is registered for the first time in the management terminal 20), the number of times of use is set to zero. In the example shown in FIG. 4, the first region 26A displays the surgical instrument 82, the number of times of use of which reached the limit of the number of times of use thereof (the number of times of use is 10, and the limit of the number of times of use is 10). For example, when the identification mark 84 of the surgical instrument 82 is read by the reader 60, the updater 34 updates the number of times of sterilization of the surgical instrument 82. Namely, the updater 34 adds “1” to the number of times of sterilization. When the reader 60 reads the identification mark 84 for the first time, the number of times of sterilization is set to zero. For example, the updater 34 updates the elapsed time of the surgical instrument 82. The time point at which the identification mark 84 is read by the reader 60 for the first time is set as the start of use of the surgical instrument 82.

The first determiner 36 determines whether or not the state of use of the surgical instrument 82 meets the predetermined discard condition. More specifically, the first determiner 36 determines whether or not the state of use of the surgical instrument 82 meets any of the first through third conditions described above. When the state of use of the surgical instrument 82 is updated by the updater 34, the first determiner 36 determines whether or not the state of use of the surgical instrument 82 meets the predetermined discard condition. In the case where the information on the surgical instrument 82 includes information only on the number of times of use and the limit of the number of times of use of the surgical instrument 82, the first determiner 36 does not determine whether or not the state of use of the surgical instrument 82 meets the second condition or the third condition.

In the case where the first determiner 36 determines that the state of use of the surgical instrument 82 meets the predetermined discard condition, the first notifier 38 notifies that the surgical instrument 82 needs to be discarded. In the example shown in FIG. 5, the number of times of use of the surgical instrument 82 reached the limit of the number of times of use, and therefore, the first notifier 38 displays a dialog box 27 indicating “reached the limit of the number of times of use” on the basic operation screen 26.

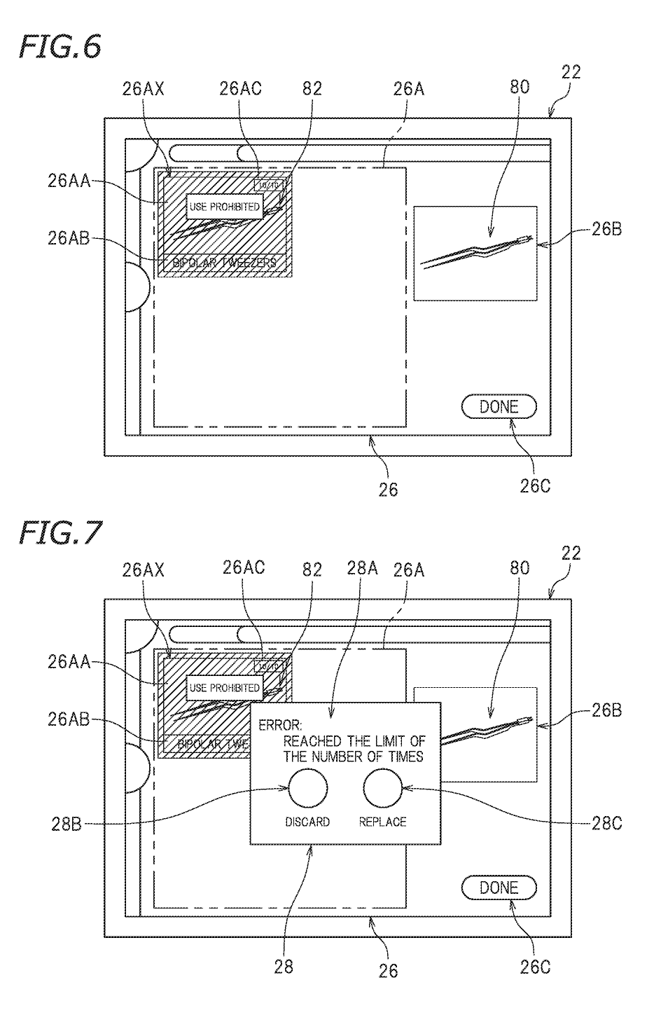

In the assembly step 94, the selector 40 selects, by use of the first region 26A, one of the surgical instruments included in the surgical instrument set 80 displayed in the second region 26B of the display device 22. The surgical instrument 82 is selected by the input device 24 by use of the first region 26A. The selector 40 is configured, in the case where the first determiner 36 determines that the state of use of a certain surgical instrument 82 meets the predetermined discard condition, not to select the surgical instrument 82 determined as meeting the predetermined discard condition. Referring to FIG. 6, in the case where the first determiner 36 determines that the state of use of the surgical instrument 82 displayed in the first region 26A meets the predetermined discard condition, it is indicated that this surgical instrument 82 is prohibited from being used, and the selector 40 cannot select this surgical instrument 82.

The first notifier 38 notifies that the surgical instrument 82 prohibited from being used needs to be discarded. In the example shown in FIG. 7, the number of times of use of the surgical instrument 82 reached the limit of the number of times of use and the surgical instrument 82 is prohibited from being used, and therefore, the first notifier 38 displays a dialog box 28 on the basic operation screen 26. The dialog box 28 includes a display region 28A indicating “error: reached the limit of the number of times of use”, a button 28B for execution of a discard process, and a button 28C for execution of a replacement process. The button 28B is pressed by the input device 24, so that the information on the surgical instrument 82 prohibited from being used is deleted from the management terminal 20. At this point, the name of the operator and the date/time of the execution of the discard process are registered in the management terminal 20. The button 28C may be pressed by the input device 24, so that a surgical instrument 82 of the same type as that of the surgical instrument 82 prohibited from being used is newly registered in the management terminal 20. At this point, the name of the operator and the date/time of the execution of the replacement process are registered in the management terminal 20.

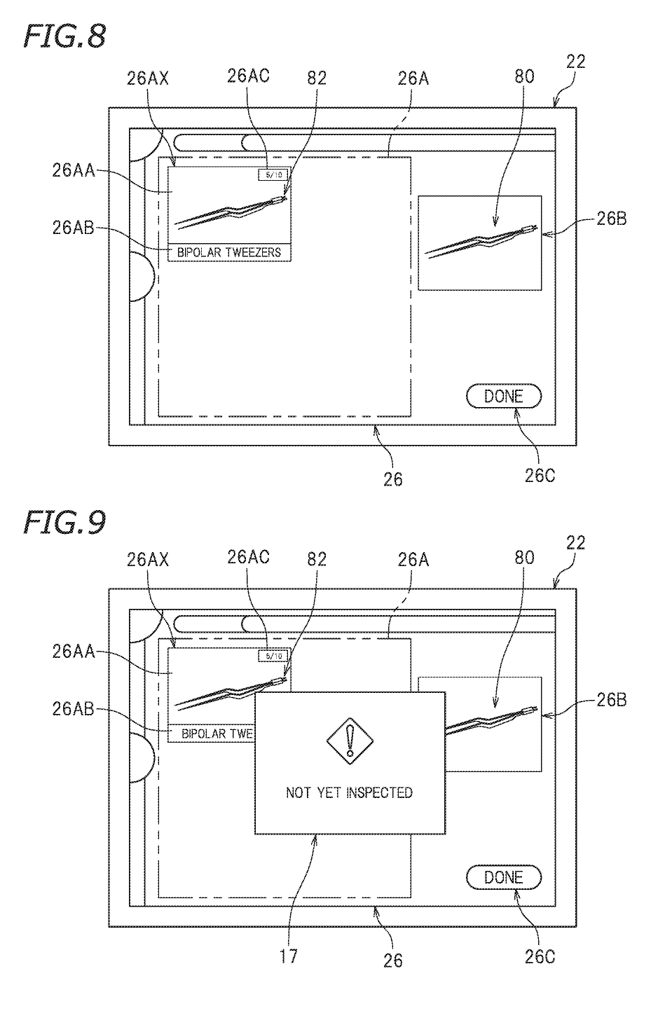

FIG. 8 shows an example of the basic operation screen 26 displayed on the display device 22 when the identification mark 84 of a surgical instrument 82 is read by the reader 60 in the assembly step 94. In the example shown in FIG. 8, the first region 26A displays the surgical instrument 82, the number of times of use of which reached half of the limit of the number of times of use thereof (i.e., the number of times of use is 5, and the limit of the number of times of use is 10).

The second determiner 42 determines whether or not the state of use of the surgical instrument 82 meets the predetermined inspection condition. More specifically, the second determiner 42 determines whether or not the state of use of the surgical instrument 82 meets any of the fourth through sixth conditions described above. When the state of use of the surgical instrument is updated by the updater 34, the second determiner 42 determines whether or not the state of use of the surgical instrument 82 meets the predetermined inspection condition. In the case where the information on the surgical instrument 82 includes information only on the number of times of use and the limit of the number of times of use of the surgical instrument 82, the second determiner 42 does not determine whether or not the state of use of the surgical instrument 82 meets the fifth condition or the sixth condition.

In the case where the second determiner 42 determines that the state of use of the surgical instrument 82 meets the predetermined inspection condition, the second notifier 44 notifies that the surgical instrument 82 needs to be inspected. In the example shown in FIG. 9, the number of times of use of the surgical instrument 82 reached half of the limit of the number of times of use, and therefore, the second notifier 44 displays a dialog box 17 indicating “not yet inspected” on the basic operation screen 26.

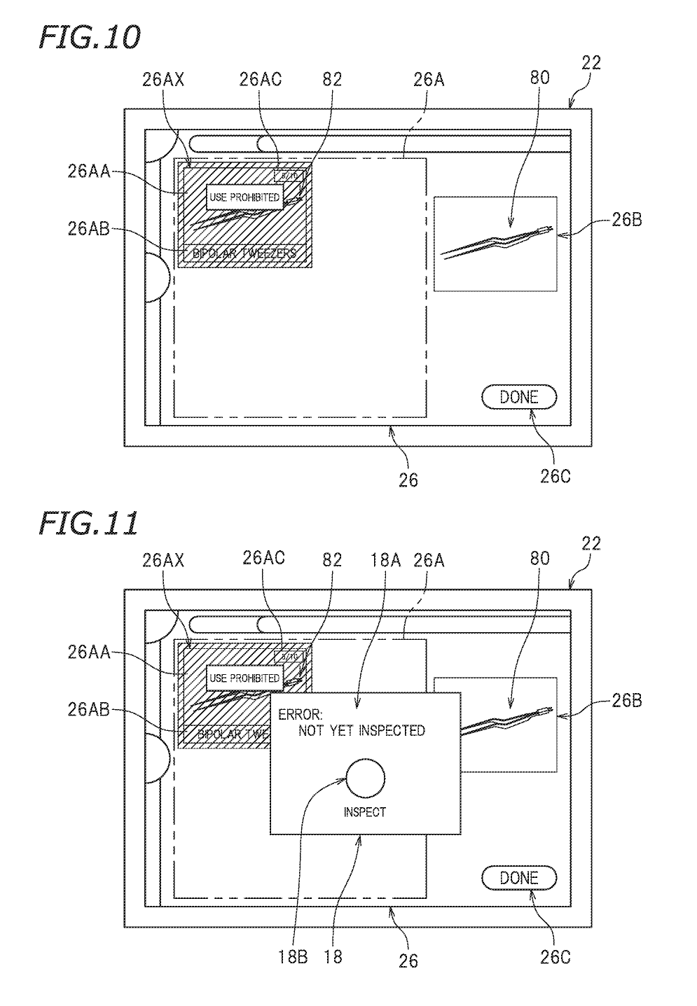

The selector 40 is configured, in the case where the second determiner 42 determines that the state of use of a certain surgical instrument 82 meets the predetermined inspection condition, not to select the surgical instrument 82 determined as meeting the predetermined inspection condition. Referring to FIG. 10, in the case where the second determiner 42 determines that the state of use of the surgical instrument 82 displayed on the first region 26A meets the predetermined inspection condition, it is indicated that this surgical instrument 82 is prohibited from being used, and the selector 40 cannot select this surgical instrument 82.

The second notifier 44 notifies that the surgical instrument 82 prohibited from being used needs to be inspected. In the example shown in FIG. 11, the number of times of use of the surgical instrument 82 reached half of the limit of the number of times of use and the surgical instrument 82 is prohibited from being used, and therefore, the second notifier 44 displays a dialog box 18 on the basic operation screen 26. The dialog box 18 includes a display region 18A indicating “error: not yet inspected” and a button 18B for execution of an inspection process. The button 18B is pressed by the input device 24, so that it is registered in the management terminal 20 that the surgical instrument 82 prohibited from being used has been inspected. At this point, the name of the operator and the date/time of the execution of the inspection process are registered in the management terminal 20. When the button 18B is pressed, an inspection procedure or the like for the surgical instrument 82 of interest may be displayed on the basic operation screen 26.

The determiner 48 determines whether or not the surgical instrument 82 has been inspected. The determiner 48 determines that the surgical instrument 82 has been inspected when, for example, the button 18B is pressed. In the case where the determiner 48 determines that the surgical instrument 82 has been inspected, the message “use prohibited” regarding the surgical instrument 82 is erased. As a result, the selector 40 is allowed to select the inspected surgical instrument 82.

The changer 46 is configured to change the discard condition stored on the storage 32. The post-change discard condition changed by the changer 46 is stored on the storage 32. In the case where the discard condition is changed, the first determiner 36 determines whether or not the state of use of the surgical instrument 82 meets the post-change discard condition. In the case where the first determiner 36 determines that the state of use of the surgical instrument 82 meets the post-change discard condition, the first notifier 38 notifies that the surgical instrument 82 needs to be discarded. The changer 46 is configured to change the inspection condition stored on the storage 32. The post-change inspection condition changed by the changer 46 is stored on the storage 32. In the case where the inspection condition is changed, the second determiner 42 determines whether or not the state of use of the surgical instrument 82 meets the post-change inspection condition. In the case where the second determiner 42 determines that the state of use of the surgical instrument 82 meets the post-change inspection condition, the second notifier 44 notifies that the surgical instrument 82 needs to be inspected. The discard condition and the inspection condition may be changed by the operator when necessary. When the discard condition or the inspection condition is changed, the name of the operator and the date/time of such a change process are registered in the management terminal 20.

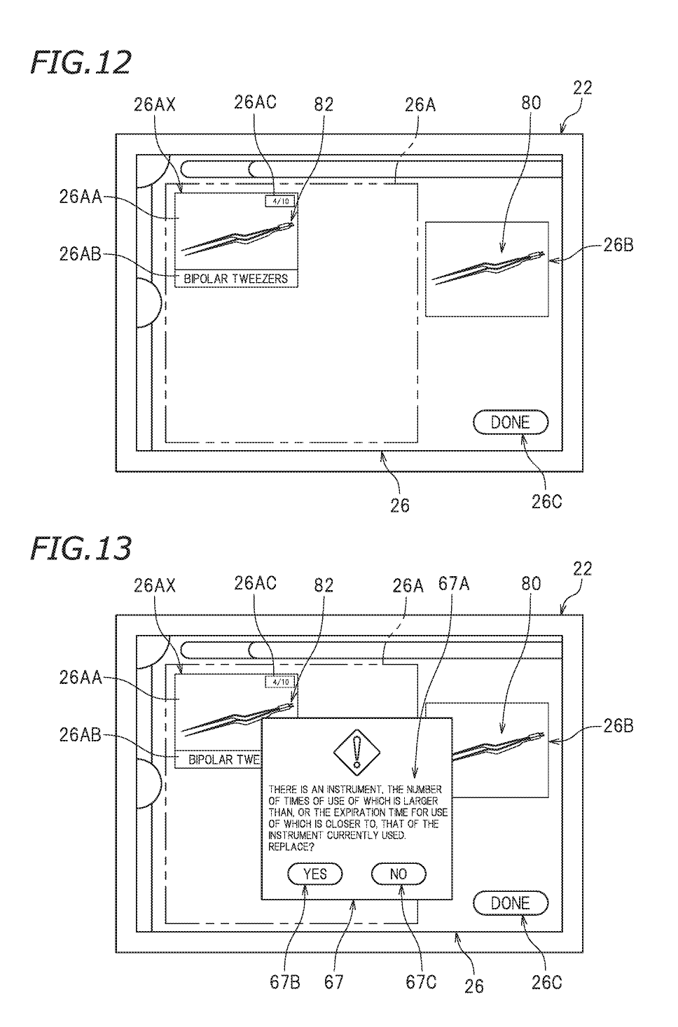

FIG. 12 shows an example of the basic operation screen 26 displayed on the display device 22 when the identification mark 84 of a surgical instrument 82 is read by the reader 60 in the assembly step 94. In the example shown in FIG. 12, the first region 26A displays the surgical instrument 82, the number of times of use of which is 4. Now, an example in which the surgical instrument 82, the number of times of use of which is 4 times, is used to assemble the surgical instrument set 80 by the selector 40 will be described.

The proposer 50 proposes changing the surgical instrument 82 selected by the selector 40 to another specific surgical instrument 82. The surgical instrument 82 selected by the selector 40 and the another specific surgical instrument 82 proposed by the proposer 50 are of the same type. The proposer 50 proposes changing the surgical instrument 82 selected by the selector 40 to the another specific surgical instrument 82 based on the state of use of the surgical instrument 82 selected by the selector 40 and the state of use of each of the plurality of surgical instruments 82 managed by the management terminal 20. The proposer 50 proposes changing the surgical instrument 82 selected by the selector 40 to, for example, a certain surgical instrument, the number of times of use of which is larger than the number of times of use of the surgical instrument 82 selected by the selector 40, the certain surgical instrument being proposed as the specific surgical instrument 82. The proposer 50 may propose changing the surgical instrument 82 selected by the selector 40 to, for example, a certain surgical instrument 82, the number of times of sterilization of which in the sterilization step is larger than the number of times of sterilization of the surgical instrument 82 selected by the selector 40, the certain surgical instrument 82 being proposed as the specific surgical instrument 82. The proposer 50 may propose changing the surgical instrument 82 selected by the selector 40 to, for example, a certain surgical instrument 82, the elapsed time period after the start of use of which is longer than the elapsed time period after the start of use of the surgical instrument 82 selected by the selector 40, the certain surgical instrument 82 being proposed as the specific surgical instrument.

In the example shown in FIG. 13, the proposer 50 displays a dialog box 67 on the basic operation screen 26. The dialog box 67 includes a display region 67A indicating “There is an instrument, the number of times of use of which is larger than, or the expiration time for use of which is closer than, that of the instrument currently used. Replace?”, a button 67B for execution of the replacement, and a button 67C for non-execution of the replacement. (The “instrument currently used” mentioned above is the instrument selected by the selector 40.) When the button 67C is pressed by the input device 24, the surgical instrument 82 is not replaced. All the surgical instruments 82 included in the surgical instrument set 80 are selected, the assembly of the surgical instrument set 80 is finished.

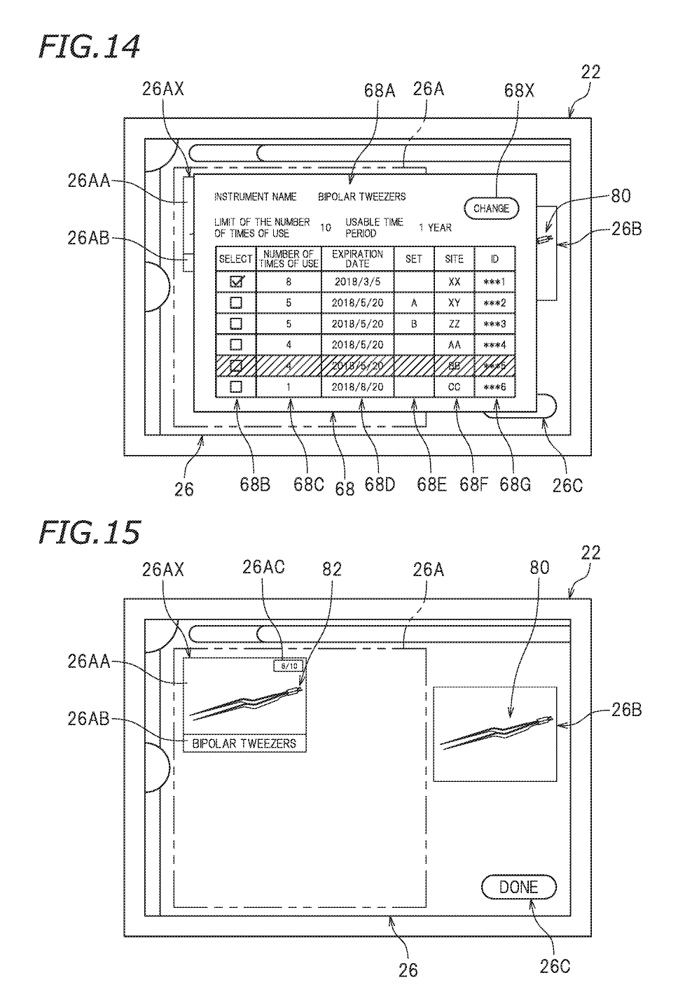

By contrast, referring to FIG. 14, when the button 67B is pressed by the input device 24, a list 68 of the surgical instruments 82 that are registered in the management terminal 20 and are of the same type as that of the surgical instrument 82 selected by the selector 40 is displayed on the basic operation screen 26. Above the list 68, there is a display region 68A, where the name, the number of times of use, and usable time period of the instrument selected by the selector 40 are displayed. The list 68 includes a first display column 68B, which shows a check box for each of the surgical instruments 82, a second display column 68C, in which the number of times of use of each of the surgical instruments 82 is recorded, a third display column 68D, in which the expiration date for use of each of the surgical instruments 82 is recorded, a fourth display column 68E, in which the name of each of the surgical instrument sets 80 is recorded, a fifth display column 68F, in which the storage site of each of the surgical instruments 82 is recorded, and a sixth display column 68G, in which the ID of each of the surgical instruments 82 is recorded. Regarding the fourth display column 68E, in the case where the surgical instrument 82 is already used for a surgical instrument set 80, the name of the surgical instrument set 80 is recorded. In the case where the surgical instrument 82 has not been used for any surgical instrument set 80, no name is recorded. In an area above and to the right of the list 68, a button 68X to be pressed when it is determined to change the surgical instrument 82 selected by the selector 40 to the surgical instrument 82 for which the check box in the first display column 68B is checked. In the example shown in FIG. 14, the surgical instrument 82 selected by the selector 40 is hatched (instrument having the ID of XXX5), and the check box of the surgical instrument 82, the number of times of use of which is 8, is checked. In the example shown in FIG. 14, the button 68X is pressed by the input device 24, so that the surgical instrument 82, the number of times of use of which is 4, is changed to the surgical instrument 82, the number of times of use of which is 8 (instrument having the ID of XXX1). As shown in FIG. 15, the first region 26A of the basic operation screen 26 displays the post-change surgical instrument 82, namely, the surgical instrument 82, the number of times of use of which is 8.

As described above, with the surgical instrument management system 10 according to this preferred embodiment, the updater 34 of the management terminal 20 updates the state of use of the surgical instrument 82 provided with the identification mark 84. The first determiner 36 of the management terminal 20 determines whether or not the state of use of the surgical instrument 82 meets the discard condition, stored on the storage 32, under which the surgical instrument 82 is to be discarded. In the case where the first determiner 36 determines that the state of use of the surgical instrument 82 meets the discard condition, the first notifier 38 of the management terminal 20 notifies that the surgical instrument 82 needs to be discarded. In this manner, the state of use of the surgical instrument 82 is updated at a predetermined timing by the management terminal 20. In the case where it is determined that the surgical instrument 82 needs to be discarded based on the state of use thereof, it is notified that the surgical instrument 82 needs to be discarded. Therefore, the operator needs to discard only the notified surgical instrument 82, and the situation where the operator forgets discarding the surgical instrument 82 to be discarded or discards the surgical instrument 82 that is still usable is prevented. The so-called “single-time use” surgical instrument 82 is opened immediately before the surgical operation and is not provided with the identification mark 84. Therefore, when, for example, a certain surgical instrument 82 is found not to be provided with the identification mark 84 when the reader 60 attempts to read the identification mark 84 in the assembly step 94, the operator may recognize that this surgical instrument 82 is for “single-time use” and thus discard this surgical instrument 82.

With the surgical instrument management system 10 according to this preferred embodiment, the state of use of the surgical instrument 82 includes the number of times of use of the surgical instrument 82. The predetermined discard condition includes the first condition that the number of times of use reached the limit of the number of times of use by which the surgical instrument 82 is permitted to be used in the surgical operation step 91. With such a setting, when the number of times of use of the surgical instrument 82 is found to have reached the limit of the number of times of use, it is notified to the operator that the surgical instrument 82 needs to be discarded. In the case where a limit is provided on the number of times of use of the surgical instrument 82, the timing to discard the surgical instrument 82 may be managed properly.

With the surgical instrument management system 10 according to this preferred embodiment, the updater 34 updates the number of times of use when the identification mark 84 is read by the reader 60 in the assembly step 94. In this manner, the number of times of use of the surgical instrument 82 is managed in the assembly step 94. Therefore, the situation is prevented where the surgical instrument 82 to be discarded is used to assemble the surgical instrument set 80, where the surgical instrument 82 to be discarded is sterilized in the sterilization step 95, or where the surgical instrument 82 to be discarded is used for the surgical operation in the surgical operation step 91.

With the surgical instrument management system 10 according to this preferred embodiment, the state of use of the surgical instrument 82 includes the number of times of sterilization of the surgical instrument 82 in the sterilization step 95. The predetermined discard condition includes the second condition that the number of times of sterilization reached the limit of the number of times of sterilization by which the surgical instrument 82 is permitted to be sterilized in the sterilization step 95. With such a setting, when the number of times of sterilization of the surgical instrument 82 is found to have reached the limit of the number of times of sterilization, it is notified to the operator that the surgical instrument 82 needs to be discarded. In the case where a limit is provided on the number of times of sterilization of the surgical instrument 82, the timing to discard the surgical instrument 82 may be managed properly. In the case where the sterilized surgical instrument set 80 is not used for a long time period, the effect of sterilization expires and thus the surgical instrument set 80 needs to be sterilized again. There is a case where the sterilized surgical instrument set 80 is opened and the surgical instruments 82 are re-assembled. In such cases, the surgical instrument 82 has not been used in the surgical operation step 91, and therefore, is not properly managed based on the number of times of use thereof.

With the surgical instrument management system 10 according to this preferred embodiment, the updater 34 updates the number of times of sterilization when the identification mark 84 is read by the reader 60 in the assembly step 94. In this manner, the number of times of sterilization of the surgical instrument 82 is managed in the assembly step 94. Therefore, the situation is prevented where the surgical instrument 82 to be discarded is used to assemble the surgical instrument set 80, where the surgical instrument 82 to be discarded is sterilized in the sterilization step 95, or where the surgical instrument 82 to be discarded is used for the surgical operation in the surgical operation step 91.

With the surgical instrument management system 10 according to this preferred embodiment, the state of use of the surgical instrument 82 includes the elapsed time after the start of use of the surgical instrument 82. The predetermined discard condition includes the third condition that the elapsed time period reached the usable time period, in which the surgical instrument 82 is permitted to be used. With such a setting, when the elapsed time period of the surgical instrument 82 is found to have reached the usable time period, it is notified to the operator that the surgical instrument 82 needs to be discarded. In the case where a usable time period is provided on the surgical instrument 82, the timing to discard the surgical instrument 82 may be managed properly.

With the surgical instrument management system 10 according to this preferred embodiment, the management terminal 20 includes the changer 46, which changes the predetermined discard condition. Usually, the discard condition under which the surgical instrument 82 is to be discarded is stored on the storage 32 in advance. It may become necessary, at a later time point, to change the discard condition, for example, to decrease the limit of the number of times of use. In this preferred embodiment, the discard condition may be changed flexibly.

With the surgical instrument management system 10 according to this preferred embodiment, the second determiner 42 determines whether or not the state of use of the surgical instrument 82 meets the inspection condition, stored on the storage 32, under which the surgical instrument 82 is to be inspected. In the case where the second determiner 42 determines that the state of use of the surgical instrument 82 meets the inspection condition, the second notifier 44 of the management terminal 20 notifies that the surgical instrument 82 needs to be inspected. In this manner, the state of use of the surgical instrument 82 is recorded constantly in the management terminal 20. In the case where it is determined that the surgical instrument 82 needs to be inspected based on the state of use thereof, it is notified that the surgical instrument 82 needs to be inspected. Therefore, the operator does not need to keep in mind the time to inspect the surgical instrument 82, and merely needs to inspect the notified surgical instrument 82. This alleviates the work load of the operator.

With the surgical instrument management system 10 according to this preferred embodiment, the management terminal 20 includes the selector 40, which selects the surgical instrument 82 included in the surgical instrument set 80 in the assembly step 94. The selector 40 is configured not to select the surgical instrument 82 determined, by the second determiner 42, as meeting the predetermined inspection condition. With such a setting, an uninspected surgical instrument 82 is prevented from being used in the surgical operation step 91.

With the surgical instrument management system 10 according to this preferred embodiment, the management terminal 20 includes the determiner 48, which determines whether or not the surgical instrument 82 has been inspected. The selector 40 is configured to select the surgical instrument 82 determined, by the determiner 48, as having been inspected. The inspected surgical instrument 82 is usable with no problem. Such a surgical instrument 82 may be used to perform the surgical operation properly.

With the surgical instrument management system 10 according to this preferred embodiment, the proposer 50 of the management terminal 20 proposes changing the surgical instrument 82 selected by the selector 40 to a specific surgical instrument 82 among the plurality of surgical instruments 82 managed by the management terminal 20, based on the state use of the surgical instrument 82 selected by the selector 40 and the state of use of each of the plurality of surgical instruments 82 managed by the management terminal 20. In the case where the surgical instrument set 80 is to be assembled in the assembly step 94, it may be better to use a surgical instrument 82 more appropriate than the surgical instrument 82 selected by the selector 40 (e.g., a surgical instrument 82, the number of times of use of which is larger than, a surgical instrument 82, the number of times of sterilization of which is larger than, or a surgical instrument 82, the expiration time for use of which is closer than, that of the surgical instrument 82 selected by the selector 40). Even in such a case, the proposer 50 makes a proposal, and therefore, the operator does not need to keep in mind the state of use of all the surgical instruments 82.

With the surgical instrument management system 10 according to this preferred embodiment, the proposer 50 may propose changing the surgical instrument 82 selected by the selector 40 to a certain surgical instrument 82, the number of times of use of which is larger than the number of times of use of the surgical instrument 82 selected by the selector 40, the certain surgical instrument 82 being proposed as the specific surgical instrument 82 among the plurality of managed surgical instruments 82. With such a setting, the surgical instrument 82 is usable more efficiently.

With the surgical instrument management system 10 according to this preferred embodiment, the proposer 50 may propose changing the surgical instrument 82 selected by the selector 40 to a certain surgical instrument 82, the number of times of sterilization of which in the sterilization step is larger than the number of times of sterilization of the surgical instrument 82 selected by the selector 40, the certain surgical instrument 82 being proposed as the specific surgical instrument 82 among the plurality of managed surgical instruments 82. With such a setting, the surgical instrument 82 is usable more efficiently.

With the surgical instrument management system 10 according to this preferred embodiment, the proposer 50 may propose changing the surgical instrument 82 selected by the selector 40 to a certain surgical instrument 82, the elapsed time period of which is longer than the elapsed time period of the surgical instrument 82 selected by the selector 40, the certain surgical instrument 82 being proposed as the specific surgical instrument 82 among the plurality of managed surgical instruments 82. With such a setting, the surgical instrument 82 is usable more efficiently.

In the above-described preferred embodiment, the first condition included in the discard condition is that the number of times of use of the surgical instrument 82 reached the limit of the number of times of use. The present invention is not limited to this. The first condition may be that the number of times of use of the surgical instrument 82 exceeded the limit of the number of times of use. The second condition included in the discard condition is that the number of times of sterilization of the surgical instrument 82 reached the limit of the number of times of sterilization. The present invention is not limited to this. The second condition may be that the number of times of sterilization of the surgical instrument 82 exceeded the limit of the number of times of sterilization. The third condition included in the discard condition is that the elapsed time period of the surgical instrument 82 reached the usable time period. The present invention is not limited to this. The third condition may be that the elapsed time period of the surgical instrument 82 exceeded the usable time period.

In the above-described preferred embodiment, the fourth condition included in the inspection condition is that the number of times of use of the surgical instrument 82 reached a predetermined number smaller than the limit of the number of times of use. The present invention is not limited to this. The fourth condition may be that the number of times of use of the surgical instrument 82 exceeded a predetermined number smaller than the limit of the number of times of use. The fifth condition included in the inspection condition is that the number of times of sterilization of the surgical instrument 82 reached a predetermined number smaller than the limit of the number of times of sterilization. The present invention is not limited to this. The fifth condition may be that the number of times of sterilization of the surgical instrument 82 exceeded a predetermined number smaller than the limit of the number of times of sterilization. The sixth condition included in the inspection condition is that the elapsed time period of the surgical instrument 82 reached a predetermined time period shorter than the usable time period. The present invention is not limited to this. The sixth condition may be that the elapsed time period of the surgical instrument 82 exceeded a predetermined time period shorter than the usable time period.

There may be a case where the elapsed time period of a certain surgical instrument 82 included in the surgical instrument set 80 had not reached the usable time period at the time when the surgical instrument set 80 was assembled in the assembly step, but reached the usable time period in the storage step 96. The controller 30 of the management terminal 20 may include a display that, in such a case, displays that the elapsed time period of the surgical instrument 82 reached the usable time period.

In the above-described preferred embodiment, the state of use of the surgical instrument 82 is updated in the assembly step 94. Alternatively, the state of use of the surgical instrument 82 may be updated in a step, in the circulation cycle 90, other than the assembly step 94. For example, the state of use of the surgical instrument 82 may be updated before the surgical instrument 82 is cleaned in the cleaning step 93. With such a setting, the surgical instrument 82 to be discarded does not need to be cleaned.

The controller 30 of the management terminal 20 may include a first selector that, when the plurality of surgical instruments 82 are to be used to assemble the surgical instrument set 80 in the assembly step 94, automatically selects, for example, the surgical instruments 82, the numbers of times of use of which are close to each other (e.g., the instruments, the numbers of times of use of which are 0 to 3). The controller 30 of the management terminal 20 may include a second selector that, when the plurality of surgical instruments 82 are to be used to assemble the surgical instrument set 80 in the assembly step 94, automatically selects, for example, the surgical instruments 82 to be used, sequentially from the surgical instrument 82, the expiration time for use is closest.

The terms and expressions used herein are for description only and are not to be interpreted in a limited sense. These terms and expressions should be recognized as not excluding any equivalents to the elements shown and described herein and as allowing any modification encompassed in the scope of the claims. The present invention may be embodied in many various forms. This disclosure should be regarded as providing preferred embodiments of the principles of the present invention. These preferred embodiments are provided with the understanding that they are not intended to limit the present invention to the preferred embodiments described in the specification and/or shown in the drawings. The present invention is not limited to the preferred embodiments described herein. The present invention encompasses any of preferred embodiments including equivalent elements, modifications, deletions, combinations, improvements and/or alterations which can be recognized by a person of ordinary skill in the art based on the disclosure. The elements of each claim should be interpreted broadly based on the terms used in the claim, and should not be limited to any of the preferred embodiments described in this specification or used during the prosecution of the present application.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A surgical instrument management system managing a state of use of a surgical instrument that includes an identification mark and is usable repeatedly in a predetermined circulation cycle including performing a surgical operation using the surgical instrument, collecting the surgical instrument used for the surgical operation, cleaning the collected surgical instrument, assembling a surgical instrument set including the surgical instrument, sterilizing the assembled surgical instrument set, and storing the sterilized surgical instrument set, the surgical instrument management system comprising:
a reader that reads the identification mark; and
a management terminal that manages predetermined information associated with the identification mark, the predetermined information including a state of use of the surgical instrument; wherein
the management terminal includes:
a storage that stores the predetermined information and a predetermined discard condition under which the surgical instrument is to be discarded;
an updater that updates the state of use of the surgical instrument;
a first determiner that determines whether or not the state of use of the surgical instrument meets the discard condition; and
a first notifier that, in a case where the first determiner determines that the state of use of the surgical instrument meets the discard condition, notifies that the surgical instrument needs to be discarded.

2. The surgical instrument management system according to claim 1, wherein
the state of use of the surgical instrument includes a number of times of use of the surgical instrument;
the predetermined discard condition includes a first condition that the number of times of use of the surgical instrument reached, or exceeded, a limit of the number of times of use of the surgical instrument is permitted to be used in the surgical operation step;
the updater updates the number of times of use of the surgical instrument;
the first determiner determines whether or not the state of use of the surgical instrument meets the first condition; and
in a case where the first determiner determines that the state of use of the surgical instrument meets the first condition, the first notifier notifies that the surgical instrument needs to be discarded.

3. The surgical instrument management system according to claim 2, wherein the updater updates the number of times of use of the surgical instrument when the identification mark is read by the reader.

4. The surgical instrument management system according to claim 1, wherein
the state of use of the surgical instrument includes a number of times that the surgical instrument is sterilized;
the predetermined discard condition includes a second condition that the number of times that the surgical instrument is sterilized is reached, or exceeded, a limit of the number of times that the surgical instrument is permitted to be sterilized;
the updater updates the number of times that the surgical instrument is sterilized;
the first determiner determines whether or not the state of use of the surgical instrument meets the second condition; and
in a case where the first determiner determines that the state of use of the surgical instrument meets the second condition, the first notifier notifies that the surgical instrument needs to be discarded.

5. The surgical instrument management system according to claim 4, wherein the updater updates the number of times that the surgical instrument is sterilized when the identification mark is read by the reader.

6. The surgical instrument management system according to claim 1, wherein
the state of use of the surgical instrument includes an elapsed time period after start of use of the surgical instrument;
the predetermined discard condition includes a third condition that the elapsed time period reached, or exceeded, a usable time period in which the surgical instrument is permitted to be used;
the updater updates the elapsed time period of the surgical instrument;
the first determiner determines whether or not the state of use of the surgical instrument meets the third condition; and
in the case where the first determiner determines that the state of use of the surgical instrument meets the third condition, the first notifier notifies that the surgical instrument needs to be discarded.

7. The surgical instrument management system according to claim 1, wherein
the management terminal includes a changer that changes the predetermined discard condition;
the storage stores a post-change predetermined discard condition changed by the changer;
the first determiner determines whether or not the state of use of the surgical instrument meets the post-change predetermined discard condition; and
in a case where the first determiner determines that the state of use of the surgical instrument meets the post-change predetermined discard condition, the first notifier notifies that the surgical instrument needs to be discarded.

8. The surgical instrument management system according to claim 1, wherein
the storage stores a predetermined inspection condition under which the surgical instrument is to be inspected; and
the management terminal includes:
a second determiner that determines whether or not the state of use of the surgical instrument meets the predetermined inspection condition; and
a second notifier that, in a case where the second determiner determines that the state of use of the surgical instrument meets the inspection condition, notifies that the surgical instrument needs to be inspected.

9. The surgical instrument management system according to claim 8, wherein
the management terminal includes a selector that selects the surgical instrument included in the surgical instrument set; and
the selector does not select the surgical instrument determined, by the second determiner, as meeting the predetermined inspection condition.

10. The surgical instrument management system according to claim 9, wherein
the management terminal includes a determiner that determines whether or not the surgical instrument has been inspected; and
the selector selects the surgical instrument determined, by the determiner, as having been inspected.

11. The surgical instrument management system according to claim 1, wherein
the storage stores the state of use of each of a plurality of surgical instruments managed by the management terminal; and
the management terminal includes:
a selector that selects the surgical instrument included in the surgical instrument set; and
a proposer that proposes changing the surgical instrument selected by the selector to a specific surgical instrument among the plurality of surgical instruments managed by the management terminal, based on the state of use of the surgical instrument selected by the selector and the state of use of each of the plurality of managed surgical instruments.

12. The surgical instrument management system according to claim 11, wherein the proposer proposes changing the surgical instrument selected by the selector to a certain surgical instrument, the number of times of use of the certain surgical instrument being larger than the number of times of use of the surgical instrument selected by the selector, the certain surgical instrument being proposed as the specific surgical instrument among the plurality of managed surgical instruments.

13. The surgical instrument management system according to claim 11, wherein the proposer proposes changing the surgical instrument selected by the selector to a certain surgical instrument, the number of times of sterilization of the certain surgical instrument in the sterilization step being larger than the number of times of sterilization of the surgical instrument selected by the selector, the certain surgical instrument being proposed as the specific surgical instrument among the plurality of managed surgical instruments.

14. The surgical instrument management system according to claim 11, wherein the proposer proposes changing the surgical instrument selected by the selector to a certain surgical instrument, the elapsed time period after the start of use of the certain surgical instrument being longer than the elapsed time period after the start of use of the surgical instrument selected by the selector, the certain surgical instrument being proposed as the specific surgical instrument among the plurality of managed surgical instruments.
